(19) United States
(12) Patent Application Publication
(10) Pub. No.: US 2026/0245444 A1
(43) Pub. Date: Aug. 20, 2026

(54) SYSTEMS AND METHODS FOR PREDICTING A DEFINED MOTOR STATE FROM AUGMENTED REALITY HEADSET DATA FOR AUTOMATIC CUE ACTIVATION

(71) Applicant: Strolll Limited, Aldridge Walsall (GB)

(72) Inventors: Melvyn ROERDINK, Amsterdam (NL); Bert COOLEN, Amsterdam (NL); Daphne J GEERSE, Amsterdam (NL); Andreas AFFERTSHOFER, Amsterdam (NL); Jacobus J VAN HILETN, Amsterdam (NL)

(21) Appl. No.: 18/997,450

(22) PCT Filed: Jul. 10, 2023
(86) PCT No.: PCT/IB2023/057068

§ 371 Date: Jan. 21, 2025

(30) Foreign Application Priority Data
Jul. 10, 2022 (GB) 2210108.3
Jul. 20, 2022 (GB) 2210640.5

(51) Int. Cl. G08B 21/04; A61B 5/00; A61B 5/11

(52) U.S. Cl. CPC G08B 21/0446; A61B 5/112; A61B 5/6803

(57) ABSTRACT

An aspect of the invention provides a method for predicting a defined motor state from augmented reality headset data for automatic cue activation, the method comprising: receiving from at least one sensor of an augmented reality device: i) motion data corresponding to motion of a user; and ii) environmental data relating to the user's location; determining one or more parameters from the motion data and environmental data; assigning an output value to a function of the one or more parameters of the motion data and environmental data; comparing the output value to a trained data model; based on the comparison, determining a likelihood of freezing of gait; and automatically activating one or more types of cues on or via the augmented reality device upon determining that the likelihood of freezing of gait occurring is greater than a customizable threshold.

### FIELD

Aspects of the present invention relate to systems and methods for predicting a defined motor state from augmented reality (“AR”) headset data for automatic cue activation. Certain embodiments of the disclosure relate to systems and methods for predicting freezing of gait (“FOG”) in individuals suffering from neurological impairments and conditions including, but not limited to, Parkinson's disease. Upon predicting an episode of FOG, one or more types of cues may be presented to the individual to mitigate, alleviate, or prevent the occurrence of FOG. While the disclosure predominantly focuses on prediction of FOG, it will be appreciated that the claimed invention is equally applicable to predicting other motor states associated with FOG and automatically enabling cues accordingly.

### BACKGROUND

In advanced disease stages most people with Parkinson's disease (“PD”) will experience freezing of gait (“FOG”). Freezing of gait remains one of the most common debilitating aspects of Parkinson's disease and causes temporary cessation of effective stepping and a sensation of being stuck to the ground. To-date pharmacological and surgical intervention has been found to be ineffective in overcoming such symptoms. Freezing of gait can be particularly pronounced when a person seeks to make a turn, initiate walking, or walk through a confined space. Gait initiation failure or start hesitation is a component of freezing of gait which is effectively the difficulty in initiating gait. Freezing of gait negatively impacts mobility and independence and can cause confusion and emotional stress in a patient resulting in a reduced quality of life.

It has been observed that whilst people suffering with Parkinson's disease may struggle with self-directed movement such as walking, other activities which involve more goal-directed movement may remain relatively unaltered.

In response to this recognition, experiments have been performed wherein external cues are provided to persons with Parkinson's disease to assist them to prevent or overcome freezing of gait. It is known to provide a pattern of coloured stripes on the floor which provide visual cues to the person for goal-directed stepping. A physiotherapist working with a patient may, for example, use line markers on the floor which can act as a visual cue to step onto or over. The patient suffering from Parkinson's disease is encouraged to walk on these visual cue points and this strategy of goal-directed stepping has been found to be effective in modifying gait and alleviating freezing of gait.

Whilst providing physical-coloured markers on the floor has been found to be effective, it will be apparent that such an approach is relatively basic and time consuming as it requires a person physically to set up the markers and then remove the markers after the task is performed or the session is completed. Furthermore, this approach assumes that only a single person is seeking to navigate a particular space at any time. Thus, an individual's specific gait pattern is not catered for by such a basic approach and a fixed spacing between cues may be effective for some users but not others.

To address this, the applicant has previously developed an augmented reality (AR) software application that is implemented through AR glasses/headset. Such an application provides a display for providing an AR overlay over a user's field of vision which visually masks or obscures any hazards or unmapped areas which might distract, attract or confuse the user and displays a series of transverse bars or other images of visual cues which appear to the user to be located on the ground in front of the user and which act to prompt-in a goal-directed manner-the user's steps.

Visual cueing applications using AR technology have been proven to have strong potential for alleviating freezing of gait (FOG) in persons living with Parkinson's disease. Furthermore, it has been proven that visual cues work better than other cueing modalities (acoustic, haptic, etc) due to a strong sensorimotor coupling as well as a stronger action-relevance or goal-directedness. AR technology enables a user's environment to be mapped and visual cues placed on the AR overlay in action relevant locations such that the user may follow a determined safe or otherwise optimum cued path from the user's current location to a desired destination.

For example, using an AR headset, a user's environment may be mapped to determine locations where there is free space and locations where there are obstacles or hazards. Based on this environmental data, a series of visual cues may be plotted between two points. For example, in a domestic environment, the environmental data may identify that there are certain obstacles between two doors into a room. Thus, visual cues may be presented along a path between the two doors that is optimised to avoid all detected obstacles. The visual cues may be selected from a variety of different visual cues saved in a software library.

Existing AR applications for alleviating FOG generally require a user to activate visual cues when required. This may be achieved by pressing a button, issuing a voice command, or making a gesture, for example. Manual activation of the cues may be difficult for some users who suffer from significant cognitive and/or motor impairment. For example, users experiencing tremors, monotonic soft speech and/or cognitive decline may not be physically able to activate the visual cues using a physical control means or voice command. Consequently, the visual cues may be activated too late once FOG has already occurred or are needed to be displayed continuously.

Thus, the present invention seeks to provide systems and methods for predicting freezing of gait, and freeze-prone motor states like standing and turning, from AR headset data for automatic cue activation.

In particular, the present invention seeks to provide a reliable and personalised system and method for activating cues via an AR headset upon determining that there is a likelihood of freezing of gait occurring. Importantly, such a determination is required to be made before freezing of gait occurs. Thus, the present invention also seeks to provide a proactive (as opposed to reactive) system and method of automatically activating one or more types of cues in accordance with a user's real-time needs.

### SUMMARY

Aspects of the present invention relate generally to the prediction of a defined motor state in real time based on data collected by an AR headset. Through use of a trained model, a motor state, i.e., sitting, standing, walking, running, freezing, may be accurately predicted some milliseconds, or even seconds, in advance through comparison of real-time motion and environmental data with baseline data generated during training of the model. Use of such a model has been found to be highly beneficial in accurately predicting FOG episodes for automatic cue activation to be implemented on the AR headset before a FOG episode occurs. Advantageously, such an approach alleviates or mitigates FOG episodes and only activates cues for a user when they are generally needed. Such an approach provides a real time, accurate, and responsive system and method for automatically activating cues in response to determining that there is a likelihood of freezing of gait occurring.

References to augmented reality, or AR, headsets herein refer to head mounted display devices that provide images to the user through a display screen positioned in front of the user's eyes. The display screen is transparent meaning that the user can see both real world objects and virtual objects. The virtual objects may be manipulated to interact in a virtual space with real world objects. Images, text, video, animations, games, etc, may be generated for display and presented to the user through an AR headset. This device enables holographic images to be presented on a surface, i.e., floor or tabletop, and in free space that is only constrained by the field of view of the AR display of the AR device.

As used herein, the term motion data is used to describe movement data and position data of the augmented reality headset and/or equivalent data recorded by sensors positioned on or about the wearer's body and connected to the augmented reality headset by way of a wired or wireless connection. For example, inertial measurement units that are integral to the AR headset may determine acceleration and orientation of the headset in 6 degrees of freedom. Cameras facing towards the wearer's face may identify eye movements. External sensors may determine kinetics or kinematics of the wearer's feet to identify festination and shuffling prior to FOG.

As used herein, the term environmental data is used to describe data concerning the wearer's immediate environment and may include pre-stored or real-time captured data concerning maps of the environment, defined locations, raycast distances, camera data, location data, geospatial data, distance from headset to floor, for example.

Augmented reality headsets tend to comprise more than one camera that may be used to accurately and repeatably map a user's environment and the orientation and other parameters of the headset without the need for additional sensors and imaging equipment. Consequently, a conventional AR headset may be used to not only map the user's environment but also determine certain parameters of the user concerning their motion and real world positioning, for example.

In one aspect of the disclosure, a method for predicting freezing of gait comprises: receiving from at least one sensor of an augmented reality device: i) motion data corresponding to motion of a user; and ii) environmental data relating to the user's location; determining one or more parameters from the motion data and environmental data; assigning an output value to a function of the one or more parameters of the motion data and environmental data; comparing the output value to a trained data model; based on the comparison, determining a likelihood of freezing of gait; and automatically activate one or more types of cues on or via the augmented reality device upon determining that the likelihood of freezing of gait occurring is greater than a customizable threshold.

As discussed above, the prior art of cueing is effective at alleviating FOG but is not capable of predicting FOG for automatic cue activation to prevent FOG. The present invention facilitates prediction of FOG in real time based upon motion and environmental data received from an augmented reality device. Such data may be processed to predict freezing of gait in advance. The present invention is user independent and can be applied to any patient without the need for further training of the model. In some embodiments, as discussed in detail below, the model may be adjusted by varying a decision boundary to reduce the false positive rate or to improve the true positive rate (i.e., there is a trade-off between the two). Furthermore, the one or more types of cues are activated automatically in accordance with the patient's real time needs.

In one embodiment, the trained data model is an echo-state network (ESN) for motor-state prediction.

In one embodiment, the output value comprises a representation of a user's motor states.

In one embodiment the method further comprises accessing a user profile to identify the user's historic motor state when walking within a pre-determined environment, wherein the step of assigning an output value to a function of the one or more parameters of the motion data and environmental data also takes into account the data accessed from the user profile.

In one embodiment the method further comprises adjusting the threshold for an individual user to control when one or more types of cues will be automatically activated for that user.

Variability of the threshold allows for sensitivity adjustment of the step of determining a likelihood of freezing of gait. Thus, the wearer of an augmented reality device or a clinician may adjust the sensitivity of automatic cue activation. This is advantageous to for example reduce instances of reliance on cues when cues are not required (i.e., lowering the number of false alarms) or to ensure that cues are always automatically activated when they are actually required (i.e., lowering the number of missed alarms).

In one embodiment the motion data is captured by: i) first mapping an environment; ii) determining a base line position of the augmented reality headset in the environment; and iii) tracking the position of the augmented reality headset in the environment as the wearer moves in the environment.

In another embodiment the motion data is captured by at least one inertial measurement unit (IMU) of the augmented reality device and includes one or more of acceleration, orientation in time and space, and direction.

In one embodiment the environmental data comprises one or more of pre-stored or real-time captured data concerning maps of the environment, defined locations, raycast distances, camera data, location data, geospatial data, distance from headset to floor.

In one embodiment the step of activating one or more types of cues on or via the augmented reality device further comprises accessing a user profile and selecting and activating one or more types of cues that are preferred by the user as indicated in the user profile.

In one embodiment the step of determining a likelihood of occurrence of freezing of gait comprises assigning a probability distribution label to each of a plurality of motor states and selecting the motor state having the highest probability and determining from the selected motor state a likelihood of occurrence of freezing of gait.

In one embodiment the selection of the one or more types of cues is made dependent on the determined motor state, or other parameters, of the user.

Another aspect of the invention provides a system for predicting freezing of gait, the system comprising: a) means for receiving from at least one sensor of an augmented reality device: i) motion data corresponding to motion of a user; and ii) environmental data relating to the user's location; b) means for determining one or more parameters from the motion data and environmental data; c) means for assigning an output value to a function of the one or more parameters of the motion data and environmental data; d) means for comparing the output value to a trained data model; e) means for, based on the comparison, determining a likelihood of occurrence freezing of gait; and f) means for automatically activating one or more types of cues on or via the augmented reality device upon determining that the likelihood of freezing of gait occurring is greater than a customizable threshold.

Further areas of applicability of the present invention will become apparent from the detailed description provided hereinafter. The detailed description and specific examples, while indicating the preferred embodiment of the invention, are intended to be given by way of example only.

### FIGURES

Aspects and embodiments of the invention will now be described by way of reference to the following figures.

FIG. 1 illustrates a flow chart of a method for predicting freezing of gait according to the disclosure.

FIG. 2 illustrates the training process for an Echo State Network according to the disclosure.

FIG. 3 illustrates receiver operating characteristic (ROC) curves for FOG prediction and how the sensitivity of FOG episode prediction varies with the decision boundary, unveiling the trade-off between true-positive rate (sensitivity) and false-positive rate.

FIGS. 4A to 4C illustrate a pre-freezing window implemented in connection with embodiments of the disclosure.

### DESCRIPTION

The following description of the preferred embodiment(s) is merely exemplary in nature and is in no way intended to limit the invention, its application, or uses.

The description of illustrative embodiments according to principles of the present invention is intended to be read in connection with the accompanying drawings, which are to be considered part of the entire written description. In the description of embodiments of the invention disclosed herein, any reference to direction or orientation is merely intended for convenience of description and is not intended in any way to limit the scope of the present invention. Relative terms such as “lower,” “upper,” “horizontal,” “vertical,” “above,” “below,” “up,” “down,” “top” and “bottom” as well as derivatives thereof (e.g., “horizontally,” “downwardly,” “upwardly,” etc.) should be construed to refer to the orientation as then described or as shown in the drawing under discussion. These relative terms are for convenience of description only and do not require that the apparatus be constructed or operated in a particular orientation unless explicitly indicated as such. Terms such as “attached,” “affixed,” “connected,” “coupled,” “interconnected,” and similar refer to a relationship wherein structures are secured or attached to one another either directly or indirectly through intervening structures, as well as both movable or rigid attachments or relationships, unless expressly described otherwise. Moreover, the features and benefits of the invention are illustrated by reference to the exemplified embodiments. Accordingly, the invention expressly should not be limited to such exemplary embodiments illustrating some possible non-limiting combination of features that may exist alone or in other combinations of features; the scope of the invention being defined by the claims appended hereto.

Embodiments of the present invention are implemented based on a trained, tested and validated supervised machine learning algorithm containing a computationally efficient Echo State Network (ESN). It will however be appreciated that use of an ESN is just one way that embodiments of the present invention may be put into effect. In essence, the ESN receives time series input data from an augmented reality headset concerning: i) the wearer's position, motion or gait cycle parameters; and ii) the wearer's environment. This input data is subjected to the machine learning algorithm to predict an occurrence of freezing of gait directly, or the likelihood thereof as estimated from other motor states.

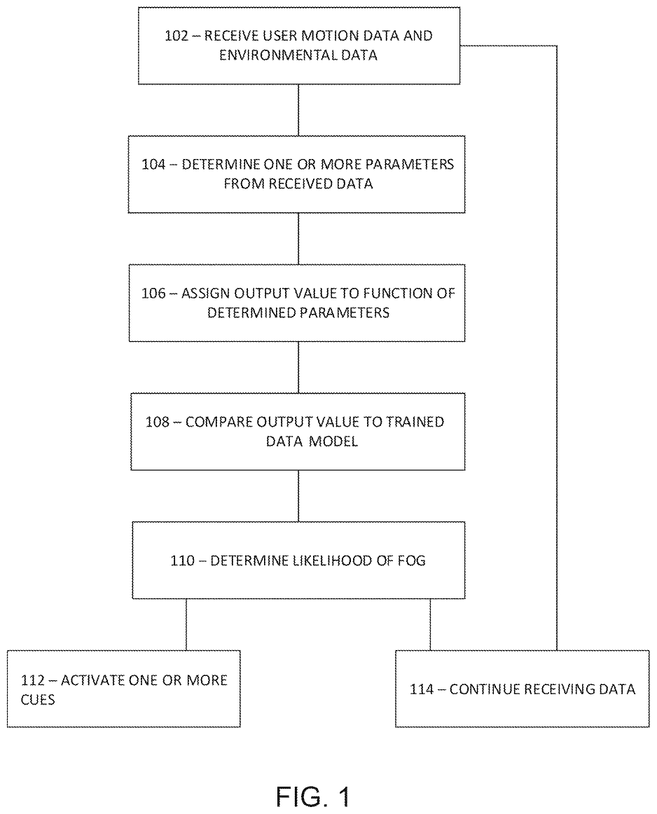

As shown in FIG. 1, a method 100 according to the disclosure is summarised. The method starts at step 102 where at least one sensor of an augmented reality device receives: i) motion data corresponding to motion of a user; and ii) environmental data relating to the user's location. At step 104 one or more parameters from the motion data and environmental data are determined. At step 106 an output value is assigned to a function of the one or more parameters of the motion data and environmental data. At step 108 the output value is compared to a trained data model. At step 110 a likelihood of occurrence of freezing of gait is determined based on the comparison. At step 112 one or more types of cues are automatically activated on or via the augmented reality device upon determining that the likelihood of freezing of gait occurring is greater than a customizable threshold. The one or more types of cues may be automatically deactivated upon conclusion of a FOG episode.

As referred to herein, one or more types of cues may be activated on or via an augmented reality device. This is intended to mean that the cues may be presented to the user directly by the augmented reality device or indirectly via the augmented reality device through external devices that may be connected to the augmented reality device by way of wired or wireless connection. Where cues are presented to the wearer by the augmented reality device, cues may be presented visually as a holographic image through the lenses of the augmented reality device, audibly through speakers of the augmented device, or through haptic means such as vibration motors of the augmented reality device. Where cues are presented to the wearer by external devices, cues may be presented visually by a projector, audibly by speakers, or through haptic means such as vibration motors, in each case positioned external to the augmented reality device. It will be appreciated that other cues may also be selected for presentation to the wearer on or via an augmented reality device.

The applicant developed a trained, tested and validated supervised machine learning algorithm by first collecting augmented reality headset data from a group of 24 persons with Parkinson's disease concerning motion and environmental data as well as video data in parallel streams. As external video data of each person was captured, the collected videos were reviewed and annotated by a pair of trained professionals in the field of Parkinson's disease to identify episodes of FOG, and other motor states, serving as ground-truth teacher streams.

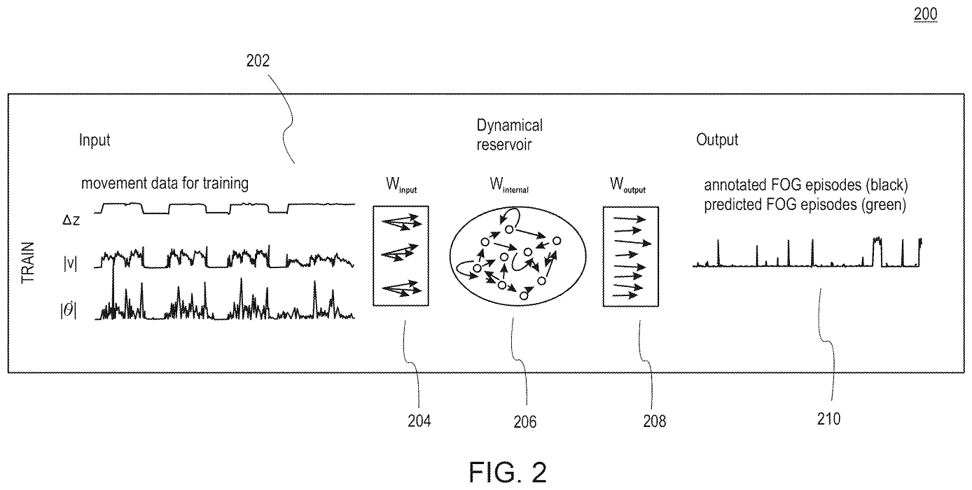

As shown in FIG. 2, the ESN transforms incoming time series of movement and environmental data 202 through a series of weights 204 into a high-dimensional state space, a dynamical reservoir 206 trained to identify specific motor states, e.g., sitting, standing, FOG, guided by ground-truth annotation streams. Once the output weights 208 are determined, the ESN output series representing motor states 208 are identified from input streams. The output series are binarized with decision boundaries or thresholds and evaluated to the ground-truth annotation streams to validate the so-identified motor states as illustrated in the output graph 210.

The sensitivity of determining an imminent episode of FOG is based on the thresholds with which the ESN is processed. The lower the threshold the higher the sensitivity and hence the higher the true positive rate (TPR; which goes at the expense of more false alarms (false positives)). Conversely, the higher the threshold the lower the sensitivity and hence the lower the false positive rate (FPR; i.e., fewer false alarms, which goes at the expense of more missed alarms (false negatives)).

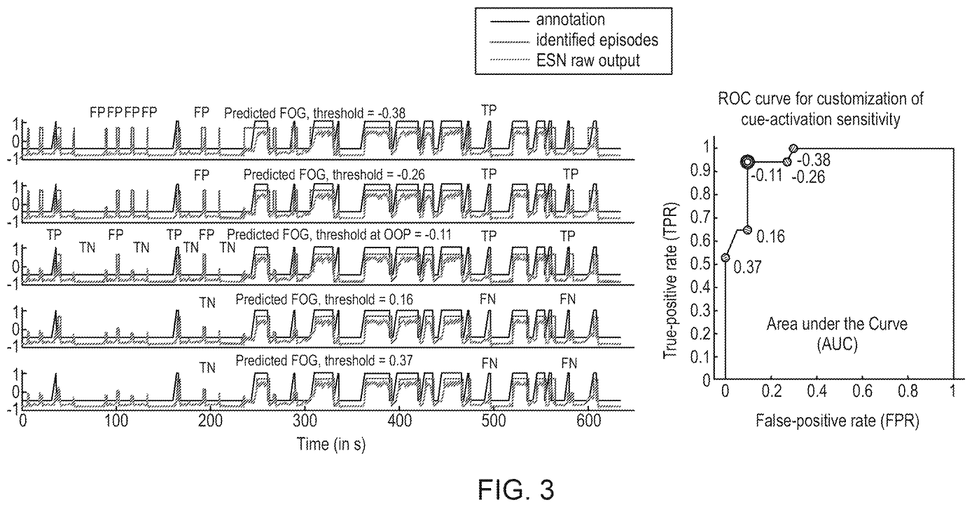

A receiver operating characteristics (ROC) curve was instigated to display this trade-off between TPR and FPR. The TPR was plotted against the FPR for various decision boundary settings. Thus, by virtue of the trade-off between TPR and FPR, by varying the decision boundary, the sensitivity of the machine learning algorithm may be adjusted by or for the wearer of the augmented reality device. A lower threshold yields more true positives at the expense of more false positives (false alarms). A higher threshold reduces the false positives, at the expense of more false negatives (missed alarms). This is illustrated in FIG. 3. Such a sensitivity adjustment may be made within the software settings of the AR device. Alternatively, sensitivity settings may be adjusted on a remote device and sent to the AR device over WiFi or Bluetooth®, for example. In one embodiment, the sensitivity settings may be adjusted by way of a holographic slider or wheel. In another embodiment, the sensitivity settings may be adjusted by way of a voice command.

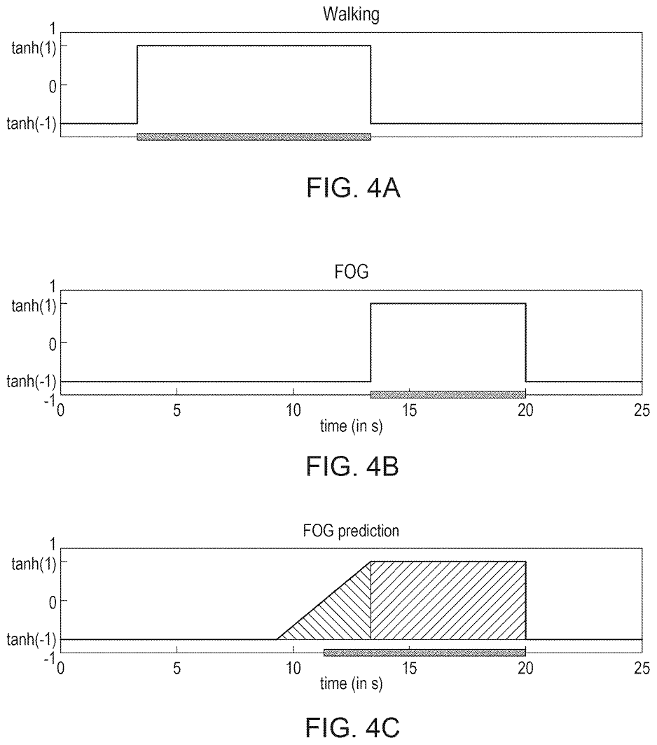

FIG. 4 illustrates a pre-freezing window in the annotation streams that may be implemented in embodiments of the invention. FIG. 4A represents the walking annotation stream for a period of time between 0 and 25. At time=0, the subject is not walking. At time=3, the subject starts walking. At time=14, the subject stops walking. At time=14 to 25, the subject is not walking.

FIG. 4B represents the freezing of gait (FOG) annotation stream for the same period of time between 0 and 25. Between time=0 and 14, the subject is not exhibiting FOG. At time=14, the subject is exhibiting FOG and continues to do so until time=20.Between time=20 and 25, the subject again is not exhibiting FOG.

FIG. 4C represents the same period of time between 0 and 25, in which the FOG annotation stream is preceded by a 5 seconds linearly increasing pre-FOG window. The green shaded area represents this pre-freezing window during which parameters of the user may be observed that indicate a likelihood that a FOG episode is about to occur. Thus, it may be determined between time=9 and 14 that a FOG episode is likely to occur. Thus, in accordance with the disclosure, one or more types of cues may be activated by way of the augmented reality headset before time=14 thus alleviating the severity of, or preventing the, predicted FOG episode.

As referred to herein, processing circuitry should be understood to mean circuitry based on one or more microcontroller units, digital signal control units, programmable logic devices, field-programmable gate arrays (FPGAs), application-specific integrated circuits (ASICs), etc. and may include a multi-core control unit (e.g., dual-core, quad-core, hexa-core or any suitable number of cores). In some embodiments processing circuitry may be distributed across multiple separate control units or processing units, for example multiple of the same type of processing units (e.g., two Intel Core i7 control units) or multiple different control units (e.g., an Intel Core i5 control unit and an Intel Core i7 control unit). In some embodiments, processing circuitry executes instructions for receiving sensor data and applying FES to a subject, wherein such instructions are stored in non-volatile memory.

In client-server-based embodiments, processing circuitry may include communication means suitable for communication with an external computing device server or other networks or servers. The instructions for carrying out the above-mentioned functionality may be stored in the non-volatile memory or on the external computing device. Processing circuitry may include a cable modem, an integrated services digital network (ISDN) modem, a digital subscriber line (DSL) modem, a telephone modem, Ethernet card, or a wireless modem for communications with other equipment, or any other suitable communications circuitry such as WiFi or Bluetooth components. Such communications may involve the Internet or any other suitable communications networks or paths. In addition, communications means may include circuitry that enables peer-to-peer communications of external computing devices, or communication of external computing devices, or communication of external computing devices in locations remote from each other.

Non-volatile memory may be embodied in an electronic storage device that is part of processing circuitry. As referred to herein, the phrase “electronic storage device” or “storage device” should be understood to mean any device for storing electronic data, computer software, or firmware, such as random-access memory, read-only memory, hard drives, flash drives, SD cards, for example.

It should be appreciated that in the above description of exemplary embodiments, various features are sometimes grouped together in a single embodiment, figure, or description thereof for the purpose of streamlining the disclosure and aiding in the understanding of or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment.

While some embodiments described herein include some, but not other features included in other embodiments, combinations of features of different embodiments are meant to be within the scope of the disclosure, and form different embodiments, as would be understood by the skilled person. For example, in the following claims, any of the claimed embodiments can be used in any combination.

Thus, while certain embodiments have been described, it will be appreciated that other and further modifications may be made thereto without departing from the spirit of the disclosure, and it is intended to cover all such modifications, enhancements, and other implementations, which fall within the true spirit and scope of this disclosure. To the maximum extent permitted by law, the scope of this disclosure is to be determined by the broadest permissible interpretation of the following claims, and shall not be restricted or limited by the foregoing detailed description

While various implementations of the disclosure have been described, it will be readily apparent to the skilled person that many more implementations are possible within the scope of the disclosure.

1. A method for predicting a defined motor state from augmented reality headset data for automatic cue activation, the method comprising:
receiving from at least one sensor of an augmented reality device: i) motion data corresponding to motion of a user; and ii) environmental data relating to the user's location;
determining one or more parameters from the motion data and environmental data;
assigning an output value to a function of the one or more parameters of the motion data and environmental data;
comparing the output value to a trained data model;
based on the comparison, determining a likelihood of occurrence of freezing of gait; and
automatically activating one or more types of cues on or via the augmented reality device upon determining that the likelihood of freezing of gait occurring is greater than a customizable threshold.

2. The method of claim 1, wherein the trained data model is an echo-state network (ESN) for motor-state prediction.

3. The method of claim 1, wherein the output value comprises a representation of a user's motor states.

4. The method of claim 3 further comprising accessing a user profile to identify the user's historic motor state when walking within a pre-determined environment, wherein the step of assigning an output value to a function of the one or more parameters of the motion data and environmental data also takes into account the data accessed from the user profile.

5. The method of claim 4 further comprising adjusting the threshold to control the sensitivity of automatic cue activation for that user.

6. The method of claim 1, wherein the motion data is captured by at least one sensor measurement series of the augmented reality device and includes one or more of position, acceleration, orientation in space, and direction data streams.

7. The method of claim 6, wherein the motion data is captured by: i) t mapping an environment; ii) determining a base line position of the augmented reality headset in the environment; and iii) tracking the position of the augmented reality headset in the environment as the wearer moves in the environment.

8. The method of claim 6, wherein the motion data is captured by at least one inertial measurement unit (IMU) of the augmented reality device and includes one or more of acceleration, orientation in time and space, and direction.

9. The method of claim 1, wherein the environmental data comprises one or more of pre-stored or real-time captured data concerning maps of the environment, defined locations, raycast distances, camera data, location data, geospatial data, distance from headset to floor.

10. The method of claim 1, wherein the step of activating one or more types of cues on or via the augmented reality device further comprises accessing a user profile and selecting and activating one or more types of cues that are preferred by the user as indicated in the user profile.

11. The method of claim 1, wherein the step of determining a likelihood of occurrence of freezing of gait comprises assigning a probability distribution label to each of a plurality of motor states and selecting the motor state having the highest probability and determining from the selected motor state a likelihood of occurrence of freezing of gait.

12. The method of claim 8, wherein the selection of the one or more types of cues is made dependent on the determined motor state, or other parameters, of the user.

13. The method of claim 12, wherein the one or more types of cues are selected from visual cues, audible cues, haptic cues, or other cues.

14. A system for predicting a defined motor state from augmented reality headset data for automatic cue activation, the system comprising:
a. means for receiving from at least one sensor of an augmented reality device:
i. motion data corresponding to motion of a user; and
ii. environmental data relating to the user's location;
b. means for determining one or more parameters from the motion data and environmental data;
c. means for assigning an output value to a function of the one or more parameters of the motion data and environmental data;
d. means for comparing the output value to a trained data model;
e. means for, based on the comparison, determining a likelihood of occurrence freezing of gait; and
f. means for automatically activating one or more types of cues on or via the augmented reality device upon determining that the likelihood of freezing of gait occurring is greater than a customizable threshold.

15. The system of claim 14, wherein the trained data model is an Echo State Network (ESN) residing in storage of the augmented reality device or via cloud storage.
